(19) United States
(12) Patent Application Publication
(10) Pub. No.: US 2019/0145978 A1
(43) Pub. Date: May 16, 2019

(54) APPLICATION OF PDE3A IN JUDGMENT OF TUMOR TREATMENT EFFECT OF ANAGRELID

(71) Applicant: Shanghai Institute of Materia Medica, Chinese Academy of Sciences, Shanghai (CN)

(72) Inventors: Qiang Yu, Shanghai (CN); Jueyu Liu, Shanghai (CN)

(73) Assignee: Shanghai Institute of Materia Medica, Chinese Academy of Sciences, Shanghai (CN)

(21) Appl. No.: 16/097,076

(22) PCT Filed: Apr. 25, 2017
(86) PCT No.: PCT/CN2017/081908

§ 371 Date: Jan. 9, 2019

(30) Foreign Application Priority Data
Apr. 26, 2016 (CN) 201610266133.2

(51) Int. Cl. G01N 33/574; C12Q 1/6886

(52) U.S. Cl. CPC G01N 33/57484; C12Q 1/6886; G01N 2333/916; C12Q 2600/158; C12Q 2600/106

(57) ABSTRACT

Application of a phosphodiesterase PDE3A and genes thereof in judgment of a tumor treatment effect of Anagrelide. Specifically provided is usage of a PDE3A gene sequence, protein, or anti-PDE3A protein specific antibody in preparation of a diagnostic reagent or a diagnostic kit. The diagnostic reagent or kit is used for (a) judging a tumor treatment effect of Anagrelide, and/or (b) judging whether Anagrelide is appropriate for treating a tumor patient. Also provided is a corresponding detection kit.

### TECHNICAL FIELD

The present invention relates to the field of tumor molecular detection. More specifically, it relates to application of PDE3A in judgment of tumor treatment effect of anagrelid.

### BACKGROUND ART

The occurrence and development of tumors are the results of multiple genes and multiple environmental factors. The diversity of the genetic background of the tumor patients determines the complexity of tumor treatment, and also indicates that individualized treatment is needed for the disease. “Targeted therapy” using targeting technology to accurately deliver drugs to tumor lesions and “target treatment” using tumor-specific gene mutations and gene functions are currently hot spots in cancer drug research. Molecular target treatment is directed to the key molecules that specifically promote tumor growth and survival in different tumor cells, and achieves an anti-tumor effect of inhibiting tumor cell growth or promoting apoptosis. Representative target drugs are shown as follows: inhibitors targeting receptor tyrosine kinases such as Gefitinib, Lapatinib, Crizotinib, etc. and the antibody drugs such as Avastin, cetuximab, etc. In addition, there are inhibitors targeting other kinases, ubiquitin-proteasome inhibitors, and histone deacetylase (HDAC) inhibitors and the like.

Unlike traditional cytotoxic chemotherapy drugs, tumor molecular targeted drugs have specific or selective anti-tumor effects and can reduce the effect of drug toxicity, which greatly improves the accuracy of tumor treatment and prolongs the survival time and improves the quality of life for patients. However, currently anti-tumor drug targets and targeted drugs are far from enough. Anagrelide is a phosphodiesterase inhibitor for the treatment of anti-thrombocytosis. Studies have shown that anagrelide or the pharmaceutically acceptable preparations thereof have new applications in inhibiting tumors, but anagrelide does not have a good effect on all types of tumors, and there is still a lack of clear diagnostic method to guide the administration of anagrelide. There is an urgent need in the art to develop biomarkers capable of judging the efficacy of anagrelide on the treatment of tumors.

### SUMMARY OF THE INVENTION

The object of the present invention is to provide a marker PDE3A for judging the effect of anagrelide on the treatment of tumors, and the use of PDE3A for judging the effect of anagrelide on the treatment of tumors.

In the first aspect of the present invention, a use of a PDE3A gene sequence, a PDE3A nucleic acid detection reagent, a PDE3A protein, and/or a PDE3A protein detection reagent is provided for preparing a diagnostic reagent or a diagnostic kit, the diagnostic reagent or kit is used for:

(a) judging the effect of anagrelide on the treatment of tumors, and/or

(b) judging whether the tumor patient is suitable for treatment with anagrelide, and/or

(c) judging the sensitivity of tumor cells to anagrelide.

In another preferred embodiment, the diagnostic reagent comprises a protein chip, a nucleic acid chip, and a combination thereof.

In another preferred embodiment, the diagnostic reagent is used in PCR, immunoblotting, and immunohistochemistry.

In another preferred embodiment, the judging comprises an auxiliary judgment and/or a prior (pre-treatment) judgment.

In another preferred embodiment, the sensitivity refers to the sensitivity of the tumor cells in the presence of anagrelide at the following concentrations: 0.001 to 0.25 μM, preferably 0.01 to 0.1 μM, more preferably 0.02 to 0.08 μM.

In another preferred embodiment, the sensitivity refers to the sensitivity of the tumor cells in the presence of anagrelide at the following concentrations: 0.25 to 10 μM, preferably 0.5 to 5 μM, more preferably 0.8 to 3 μM.

In another preferred embodiment, the sensitivity comprises the sensitivity of the tumor cells under in vitro culture conditions, and/or the sensitivity of the tumor cells in vivo.

In another preferred embodiment, the anagrelide is administered for the treatment of the tumor in an amount of 1 mg to 500 mg per day, preferably 10 mg to 250 mg per day, more preferably 30 to 100 mg per day.

In another preferred embodiment, the PDE3A gene sequence comprises an encoding sequence and/or a non-coding sequence of PDE3A.

In another preferred embodiment, the PDE3A gene sequence comprises a genomic DNA, cDNA and/or mRNA sequence.

In another preferred embodiment, the PDE3A nucleic acid detection reagent is coupled or carried with a detectable label.

In another preferred embodiment, the PDE3A protein detection reagent comprises a specific antibody against the PDE3A protein, and a protein chip.

In another preferred embodiment, the PDE3A protein detection reagent is coupled or carried with a detectable label.

In another preferred embodiment, the detectable label is selected from the group consisting of: a chromophore, a chemiluminescent group, a fluorophore, an isotope and an enzyme.

In another preferred embodiment, the PDE3A protein comprises a full length PDE3A protein, or a secreted protein thereof.

In another preferred embodiment, the PDE3A gene sequence and/or the PDE3A protein is used as a standard or control in the kit.

In another preferred embodiment, the PDE3A nucleic acid detection reagent comprises a primer, a probe, or a nucleic acid chip.

In another preferred embodiment, the tumor patient comprises a non-hematologic tumor patient or a solid tumor patient.

In another preferred embodiment, the tumor is selected from the group consisting of: lung cancer, liver cancer, gastric cancer, esophageal cancer, intestinal cancer, nasopharyngeal cancer, breast cancer, lymphoma, renal cancer, pancreatic cancer, bladder cancer, ovarian cancer, uterus cancer, bone cancer, gallbladder cancer, lip cancer, melanoma, tongue cancer, laryngeal cancer, leukemia, prostate cancer, brain cancer, squamous cell carcinoma, skin cancer, hemangioma, lipoma, thyroid cancer, glioma, cervical cancer, and a combination thereof.

In another preferred embodiment, the tumor is selected from the group consisting of: lung cancer, melanoma, renal cancer, colon cancer, glioma, and cervical cancer.

In another preferred embodiment, the diagnostic reagent is a monoclonal and polyclonal antibody, or a combination thereof.

In another preferred embodiment, the diagnostic reagent or diagnostic kit is used to detect a sample selected from the group consisting of: a surgically removed tissue sample, a biopsy puncture tissue sample, a tumor tissue lysate, a blood sample, a cell sample, a body fluid sample, a urine sample, and a combination thereof.

In another preferred embodiment, the diagnostic kit is used for detecting a section made from a tissue sample.

In another preferred embodiment, the section comprises a paraffin section, or a frozen section.

In another preferred embodiment, the detection for blood sample is to detect the expression level of PDE3A in circulating tumor cells in the peripheral blood.

In another preferred embodiment, the diagnostic kit comprises an immunoblot analysis kit and/or a PCR assay kit.

In the second aspect of the present invention, a diagnostic kit for detecting the effect of an agrelide on the treatment of tumor is provided, comprising:

(a) a first container containing a detection reagent for detecting PDE3A expression and/or PDE3A protein;

(b) a label or an instruction; and

(c) optionally a standard or reference substance, wherein the label or instruction indicates that the kit is used for (a) judging the effect of anagrelide on the treatment of tumor, and/or (b) judging whether a tumor patient is suitable for the treatment with anagrelide.

In another preferred embodiment, the detection reagent comprises: a primer, a probe, an antibody, a nucleic acid chip, a protein chip, or a combination thereof.

In another preferred embodiment, the first container comprises one or more first containers.

In another preferred embodiment, the PDE3A protein or a specific antibody thereof is coupled with or carried with a detectable label.

In another preferred embodiment, the standard or reference substance comprises a PDE3A nucleic acid sequence, a PDE3A protein, or a combination thereof.

In another preferred embodiment, the diagnostic kit further comprises a second container containing a reagent for treating a tumor tissue.

In another preferred embodiment, the diagnostic kit further contains a reagent for the immunoblot detection.

In another preferred embodiment, the label or the instruction indicates the following contents:

(i) PDE3A expression is positive in tumor tissues, and the predictions suggest that anagrelide is effective in treating the tumor, and/or the tumor patients are suitable for the treatment with anagrelide; and

(ii) PDE3A expression is negative in tumor tissues, and the predictions suggest that anagrelide is poorly effective in treating the tumor, and/or that tumor patients are not suitable for the treatment with anagrelide.

In another preferred embodiment, the positive expression of PDE3A means that the tumor cells express PDE3A and/or have PDE3A protein activity.

In another preferred embodiment, the negative expression of PDE3A means that the tumor cells have low or no expression of PDE3A and/or no PDE3A protein activity.

In another preferred embodiment, the present invention provides a use of a detection reagent for detecting a PDE3A gene sequence or protein for the preparation of a kit for (a) judging the effect of anagrelide on the treatment of a tumor, and/or (b) a marker for judging whether a tumor patient is suitable for the treatment with anagrelide.

In the third aspect of the present invention, an in vitro method for judging the sensitivity of a tumor cell to anagrelide is provided, comprising the steps of:

(i) providing a tumor cell to be tested;

(ii) detecting the expression and/or activity of PDE3A in the tumor cell to be tested in vitro, wherein if the tumor cell to be tested expresses PDE3A and/or has PDE3A protein activity, indicating that the tumor cell to be tested is sensitive to anagrelide; if the tumor cell to be tested has low or no expression of PDE3A and/or does not have PDE3A protein activity, indicating that the tumor cell to be tested is not sensitive to anagrelide.

In another preferred embodiment, the low or no expression of PDE3A means that the ratio R1 of the PDE3A mRNA level M1 in the tumor cell to the β-actin mRNA level M2 is ≤0.2, preferably ≤0.1.

In another preferred embodiment, the low or no expression of PDE3A means that the ratio R2 of PDE3A protein level P1 in the tumor cell to PDE3A protein level P2 in Bel7404 is ≤0.5, preferably ≤0.2.

In another preferred embodiment, “do not have PDE3A protein activity” means that the ratio R3 of the PDE3A activity A1 in the tumor cell to the PDE3A protein activity A2 in Bel7404 is ≤0.5, preferably ≤0.2.

In another preferred embodiment, the expression of PDE3A means that the ratio R1 of the PDE3A mRNA level M1 to the β-actin mRNA level M2 in the tumor cell is ≥0.3, preferably ≥0.5.

In another preferred embodiment, the expression of PDE3A means that the ratio R2 of PDE3A protein level P1 in the tumor cell to PDE3A protein level P2 in Bel7404 is ≥0.6, preferably ≥0.8.

In another preferred embodiment, “having PDE3A protein activity” means that the ratio R3 of the PDE3A protein activity A1 in the tumor cell to the PDE3A protein activity A2 in Bel7404 is ≥0.6, preferably ≥0.8.

In another preferred embodiment, the method further comprises: detecting the presence of any mutation in the PDE3A of the tumor cell to be tested.

In another preferred embodiment, the method further comprises:

(iii) in the presence of anagrelide, in vitro culturing the tumor cell to be tested which is determined to be sensitive to anagrelide in the previous step, and observing the growth condition of the tumor cell, thereby verifying the sensitivity of the tumor cell to anagrelide.

In another preferred embodiment, in the step (iii), the concentration of the anagrelide is 0.001 to 0.25 μM, preferably 0.01 to 0.1 μM, more preferably 0.02 to 0.08 μM.

In another preferred embodiment, in the step (iii), the concentration of the anagrelide is 0.25 to 10 μM, preferably 0.5 to 5 μM, more preferably 0.8 to 3 μM.

In another preferred embodiment, in the step (iii), “observing the growth condition of the tumor cell” includes observing the apoptosis and/or migration of the tumor cell.

In another preferred embodiment, the method is non-diagnostic and non-therapeutic.

In the fourth aspect of the present invention, a method of treating a tumor is provided, comprising the steps of: administering anagrelide to a subject in need thereof, wherein the subject is a tumor patient, and the tumor cell expresses PDE3A and/or has PDE3A protein activity.

In another preferred embodiment, the method further comprises: detecting the PDE3A expression or activity in the subject prior to administering the anagrelide.

In another preferred embodiment, the anagrelide comprises a compound of the structure of formula II, or a pharmaceutically acceptable salt or a prodrug or a derivative thereof, or a formulation thereof.

It should be understood that, within the scope of the present invention, each technical feature of the present invention described above and in the following (as examples) may be combined with each other to form a new or preferred technical solution, which is not listed here due to space limitations.

### DESCRIPTION OF FIGURE

FIG. 1 shows the expression of PDE3A protein in cell lines from different sources. Wherein α-Tubulin and GAPDH are internal reference proteins. S: anagrelide sensitive cells; M: anagrelide-moderately sensitive cells; unidentified cells are all anagrelide insensitive cells R.

FIG. 2 shows that anagrelide selectively inhibits the growth of tumor cells from different tissue sources. Wherein anagrelide (1 μM) is used for treatment for 72 hours, and MTT assay is used to detect the cell survival rate. The cells are classified by the test results: sensitive (survival rate <40%), moderately sensitive (40-75% of survival rate), resistance (survival rate >75. The cell names in the figure are marked with a circle and a triangle respectively to identify the sensitive group and the moderately sensitive group.

FIG. 3 shows the expression of PDE3A mRNA in different cells. Wherein the PDE3A mRNA level in nine cell lines is detected by RT-PCR; the ordinate: the quantitative result relative to β-actin.

FIG. 4 shows the role of PDE3A protein in tumor cell growth.

FIG. 4A shows the expression of PDE3A protein in HeLa cell by immunoblotting 48 hours after pde3a siRNA 1/2 transfection.

FIG. 4B shows the growth of HeLa cell monitored by RTCA assay before and after transfection of pde3a siRNA. Wherein, NC: negative control; KD1 and KD2: PDE3A knockdown group.

FIG. 5 shows the relationship between the expression of PDE3A protein and cytotoxicity of anagrelide.

FIG. 5A shows the cell growth curves before and after treatment with different anagrelide recorded using RTCA method, 48 hours after the transfection of siRNA into HeLa. Wherein, NC: control group, KD: PDE3A knockdown group.

FIG. 5B shows the photos taken by fluorescence microscopy after treatment of anagrelide or DMSO on HeLa cells before and after transfection for 36 hours, and PI staining for 20 minutes.

FIG. 5C shows the results of counting PI positive cells using the Cell Profiler software.

FIG. 5D shows the results of the cell survival rate measured by the MTT method, after the treatment of cells for 48 hours with anagrelide.

FIG. 6 shows the mRNA expression of other PDEs in the anagrelide-insensitive cell line, and the relative expression level obtained based on the PDE3A expression in the moderately sensitive cell Bel7404.

### DETAILED DESCRIPTION

After an extensive and in-depth study, the present inventors have firstly discovered a biomarker (PDE3A) capable of guiding the treatment of tumors with Anagrelide. Experiments have shown that if tumor tissue and peripheral blood circulating tumor cells positively express PDE3A, suggesting that anagrelide is effective in treating tumors, and/or tumor patients are suitable for treatment with anagrelide. On this basis, the inventors complete the present invention.

In particular, the present invention establishes a safe and effective diagnostic and typing method based on the expression of PDE3A in tumor cells, which can guide anagrelide clinically on individualized treatment of tumor patients in a simple, rapid and economical manner. There are currently ELISA detection kits of PDE3A on the market, however, there is no method for the diagnosis and treatment of tumors using PDE3A as a tumor marker yet. The method such as immunoblotting and PCR provided in the present invention for detecting the expression of PDE3A in tumor samples and the application of which to guide the application of anagrelide on the treatment of tumors are the first in technology and application.

The present invention has discovered for the first time that about 30% in tumor cell lines positively express PDE3A protein or mRNA, and PDE3A-positive tumor cells exhibit different degrees of sensitivity to Anagrelide, while no inhibition effect is exhibited on PDE3A-negative tumor cells with Anagrelide. Animal experiment results indicate that Anagrelide is safe at doses (10 mg-200 mg/kg, preferably 10-20 mg/kg) that inhibit tumor cell growth, therefore, Using PDE3A as a biomarker of this drug to guide the treatment of Anagrelide on tumor has theoretical basis and application value.

PDE3A Protein and Gene Sequence

As used herein, the term “PDE3A”, i.e., phosphodiesterase 3A, is a member of the serine-threonine kinase family that terminates the biological signals transmitted by cyclic adenosine monophosphate (cAMP) and cyclic guanosine monophosphate (cGMP) through hydrolyzing these second messengers in vivo, thereby regulating life activities. PDE3A is mainly distributed in heart, platelets, vascular smooth muscle and oocyte; the hydrolysis efficiency of cAMP is 10 times that of cGMP.

In the present invention, the term “the protein of the present invention”, “PDE3A protein”, or “PDE3A polypeptide” can be used interchangeably and refer to a protein or polypeptide having the amino acid sequence of human PDE3A protein (UniProtKB/Swiss-Prot: Q14432), including PDE3A protein with or without the starting methionine. In addition, the term further comprises full length of PDE3A and fragments thereof, particularly secretory fragments (or secretory proteins). The PDE3A protein referred to in the present invention includes its entire amino acid sequence, its secreted proteins, its mutants, and its functionally active fragments.

Furthermore, the PDE3A proteins of the present invention includes glycosylated and non-glycosylated proteins.

In the present invention, the terms “PDE3A gene (ID: 5139)” and “PDE3A polynucleotide” can be used interchangeably and refer to a nucleic acid sequence having a human PDE3A nucleotide sequence (NC_000012.12). It should be understood that the substitution of nucleotides in the codon is acceptable when encodes the same amino acid. It should be further understood that nucleotide conversion is also acceptable when substituted by nucleotides to produce conservative amino acid substitution.

In the case where an amino acid fragment of PDE3A is obtained, a nucleic acid sequence encoding the fragment can be constructed therefrom, and a specific probe can be designed based on the nucleotide sequence. The full length of nucleotide sequence or a fragment thereof can usually be obtained by a PCR amplification method, a recombinant method or an artificial synthetic method. For PCR amplification, primers can be designed according to the PDE3A nucleotide sequences disclosed herein, particularly open reading frame sequences, and using the commercially available cDNA libraries or the cDNA libraries prepared according to conventional methods known to those skilled in the art as templates to amplify and obtain the relevant sequences. When the sequence is long, it is often necessary to perform twice or more times of PCR amplification, and then the amplified fragments are then spliced together in the correct order.

Once the relevant sequences are obtained, the recombinant method can be used to obtain the relevant sequences in large quantities. This is usually done by cloning it into a vector, transferring it to a cell, and then isolating the relevant sequence from the proliferated host cell by conventional methods.

In addition, artificial synthetic methods can be used to synthesize related sequences, especially when the length of the fragment is short. Usually, a long sequence of fragments can be obtained by firstly synthesizing a plurality of small fragments and then connecting them.

At present, DNA sequences encoding the proteins of the present invention (or fragments, derivatives thereof) have been obtained by chemical synthesis. The DNA sequence can then be introduced into various existing DNA molecules (e.g., vectors) and cells known in the art.

The polynucleotide sequences of the present invention can be used to express or produce recombinant PDE3A polypeptides by conventional recombinant DNA techniques.

Specific Antibody

In the present invention, the terms “antibody of the present invention” and “specific antibody against PDE3A” can be used interchangeably.

The present invention further comprises polyclonal and monoclonal antibodies, particularly monoclonal antibodies, which are specific for human PDE3A polypeptides. Here, “specific” means that the antibody can bind to a human PDE3A gene product or fragment. Preferably, it refers to those antibodies that can bind to a human PDE3A gene product or fragment but do not recognize and bind to other non-related antigen molecules. Antibodies in the present invention include those molecules capable of binding to and inhibiting the human PDE3A protein, as well as those antibodies which do not affect the function of the human PDE3A protein. The invention also includes those antibodies that bind to a modified or unmodified form of the human PDE3A gene product.

The invention comprises not only intact monoclonal or polyclonal antibodies, but also antibody fragments having immunological activity, such as Fab′ or (Fab)₂ fragments; antibody heavy chains; antibody light chains; genetically engineered single-chain Fv molecules (Ladner et al., U.S. Pat. No. 4,946,778); or chimeric antibodies, such as antibodies that have murine antibody binding specificity but still retain antibody portions from humans.

Antibodies of the present invention can be prepared by a variety of techniques known to those skilled in the art. For example, a purified human PDE3A gene product or a fragment thereof that is antigenic can be administered to an animal to induce the production of the polyclonal antibody. Similarly, cells expressing the human PDE3A protein or its antigenic fragment can be used to immunize an animal to produce antibodies. The antibody of the present invention may also be a monoclonal antibody. Such monoclonal antibodies can be prepared using hybridoma technology. The antibodies of the present invention include antibodies that block the function of the human PDE3A protein and antibodies that do not affect the function of the human PDE3A protein. The various antibodies of the present invention can be obtained by conventional immunological techniques using fragments or functional regions of the human PDE3A gene product. These fragments or functional regions can be prepared by recombinant methods or synthesized using a polypeptide synthesizer. An antibody that binds to an unmodified form of the human PDE3A gene product can be produced by immunizing an animal with a gene product produced in a prokaryotic cell (such as E. Coli); an antibody that binds to a post-translational modified form (such as, glycosylated or phosphorylated protein or polypeptide) can be obtained by immunizing an animal with a gene product produced in a eukaryotic cell, such as yeast or insect cells.

Antibodies against human PDE3A protein can be used in immunohistochemistry to detect the human PDE3A protein in specimens, particularly tumor tissues.

Anagrelide and the Use Thereof

As used herein, the terms “anagrelide”, “Anagrelide”, “Agrylin”, “ANA” can be used interchangeably and refer to a compound having the structure as shown in the following formula, or a pharmaceutically acceptable salt or a prodrug, or a derivative, or a formulation form thereof:

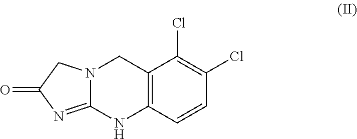 [SMILES: O=C1CN2Cc3c(ccc(Cl)c3Cl)NC2=N1]

Anagrelide is a phosphodiesterase inhibitor for the treatment of anti-thrombocytosis. Studies have shown that anagrelide or a pharmaceutically acceptable formulation thereof have new applications in inhibiting tumors. Experiments have shown that the IC50 of anagrelide inhibiting platelets in vitro is 0.27-1 μM, while anagrelide has an IC50 of less than 0.03 μM for sensitive tumor cells in vitro.

In a preferred embodiment of the present invention, the anagrelide is 6,7-dichloro-1,5-dihydroimidazo [2,1-b]quinazolin-2(3H) one.

Currently, anagrelide is clinically used as a phosphodiesterase inhibitor for the treatment of anti-thrombocytosis. Studies have shown that anagrelide or a pharmaceutically acceptable formulation thereof have new applications in inhibiting tumors, specifically including:

(a) treatment or inhibition of tumors or cancer as an anti-tumor drug;

(b) selectively inhibition of tumor cell proliferation or induction of apoptosis in vitro or in vivo;

(c) selectively regulation of the cycle of tumor cells in vitro or in vivo, and induction of cells to produce G1 cycle arrest and G2 cycle arrest.

(d) selectively inhibition of tumor cell migration in vitro or in vivo.

The anagrelide or a pharmaceutical composition thereof can be administered at a lower concentration, preferably, the anagrelide can act on a subject cell at a concentration of ≤1 μM and produce a desired effect.

Preferably, the derivative of anagrelide has the structure as shown in formula I below:

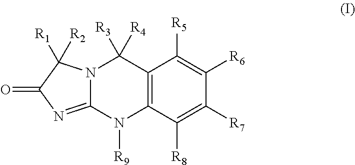

wherein,

R1 to R8 is each independently selected from the group consisting of: hydrogen atom, halogen atom, amino group, hydroxyl group, cyano group, aldehyde group, nitro group, carboxyl group (—COOH), substituted or unsubstituted C1-C10 alkyl group, substituted or unsubstituted C3-C10 cycloalkyl, substituted or unsubstituted C2-C10 alkenyl, substituted or unsubstituted C2-C10 alkynyl, substituted or unsubstituted C6-C10 aryl, substituted or unsubstituted C1-C10 heteroaryl (such as substituted or unsubstituted 5 or 6 membered heterocycle, 8 to 10 membered heteroaromatic bicyclic ring system), substituted or unsubstituted C1-C10 alkoxy group, substituted or unsubstituted C6-C10 aryl-oxy, substituted or unsubstituted C1-C10 heteroaryl-oxy, substituted or unsubstituted acyl (preferably, —CO—C1-C10 alkyl), substituted or unsubstituted ester group (preferably C1-C10 alkyl-COO—), substituted or unsubstituted C1-C10 sulfonyl group (—SO2-C1-C10 alkyl);

or R1 and R2, R3 and R4 together constitute a group selected from the group consisting of: substituted or unsubstituted C3-C20 cycloalkyl group (preferably, C3-C10 cycloalkyl group), substituted or unsubstituted C1-C20 heterocycloalkyl (preferably, substituted or unsubstituted 5 or 6 membered heterocycle, 8 to 12 membered heteroaromatic bicyclic ring system), carbonyl group (═O);

R9 is selected from the group consisting of: hydrogen atom, oxygen atom, substituted or unsubstituted C1-C10 alkyl group, substituted or unsubstituted C3-C10 cycloalkyl group, substituted or unsubstituted C6-C10 aryl group, substituted or unsubstituted C1-C10 heteroaryl, substituted or unsubstituted C1-C10 alkoxy, substituted or unsubstituted C6-C10 aryl-oxy, substituted or unsubstituted acyl group (preferably, —CO—C1-C10 alkyl), substituted or unsubstituted C1-C10 sulfonyl group;

wherein the “substituted” means that one or more hydrogen atoms on the group are substituted with a substituent selected from the group consisting of: C1-C10 alkyl, C3-C10 cycloalkyl, C1-C10 alkoxy, halogen, hydroxy, carboxy (—COOH), C1-C10 aldehyde group, C2-C10 acyl group, C2-C10 ester group, amino, phenyl group;

The phenyl group includes an unsubstituted phenyl group or a substituted phenyl group having 1-3 substituents selected from the group consisting of: halogen, C1-C10 alkyl group, cyano group, OH, nitro group, C3-C10 cycloalkyl, C1-C10 alkoxy, amino.

In another preferred embodiment, each of R1 to R8 is independently selected from the group consisting of: hydrogen atom, halogen atom, amino, hydroxyl, cyano, nitro, amino, aldehyde group, carboxyl, substituted or unsubstituted C1-C5 alkyl, substituted or unsubstituted C3-C6 cycloalkyl, substituted or unsubstituted C2-C5 alkenyl, substituted or unsubstituted C2-C5 alkynyl, substituted or unsubstituted C6-C10 aryl, substituted or unsubstituted C1-C6 heteroaryl, substituted or unsubstituted C1-C5 alkoxy, substituted or unsubstituted C6-C10 aryl-oxy, substituted or unsubstituted C1-C6 heteroaryl-oxy, substituted or unsubstituted —CO—C1-C5 alkyl, substituted or unsubstituted C1-C5 alkyl-COO—, substituted or unsubstituted C1-C5 sulfonyl; or R1 and R2, R3 and R4 together constitute a group selected from the group consisting of: substituted or unsubstituted C3-C10 cycloalkyl, substituted or unsubstituted C1-C10 heterocycloalkyl, carbonyl;

R9 is selected from the group consisting of: hydrogen atom, oxygen atom, substituted or unsubstituted C1-C5 alkyl, substituted or unsubstituted C3-C6 cycloalkyl, substituted or unsubstituted C6-C10 aryl, substituted or unsubstituted C1-C10 heteroaryl, substituted or unsubstituted C1-C5 alkoxy, substituted or unsubstituted C6-C10 aryl-oxy, substituted or unsubstituted —CO—C1-C5 alkyl, substituted or unsubstituted C1-C5 sulfonyl;

wherein, the definition of substitution is as described above.

In another preferred embodiment, R1 to R8 is each independently selected from the group consisting of: hydrogen atom, halogen atom, cyano, substituted or unsubstituted C1-C5 alkyl, substituted or unsubstituted C3-C6 cycloalkyl;

or R1 and R2, R3 and R4 together constitute a group selected from the group consisting of: substituted or unsubstituted C1-C5 cycloalkyl, substituted or unsubstituted C1-C5 heterocycloalkyl, carbonyl;

R9 is selected from the group consisting of: hydrogen atom, substituted or unsubstituted C1-C5 alkyl, substituted or unsubstituted C3-C6 cycloalkyl;

wherein, the definition of substitution is as described above.

In another preferred embodiment, 1 to 8 of the R1 to R9 is each a hydrogen atom, and preferably 2 to 7 of which is each a hydrogen atom.

In another preferred embodiment, 1 to 8 of the R1 to R8 is each a halogen atom.

In another preferred embodiment, 1 to 8 of the R1 to R8 is each a halogen atom, and the rest of the R1 to R8 is each a hydrogen atom.

In another preferred embodiment, the compound of formula I has the structure as shown in formula II:

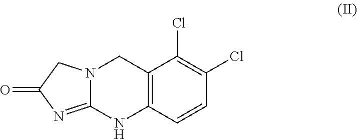 [SMILES: O=C1CN2Cc3c(ccc(Cl)c3Cl)NC2=N1]

In another preferred embodiment, the pharmaceutically acceptable salt is a salt selected from the group consisting of: hydrochloride, acetate, phosphate, and a combination thereof.

Detection Scheme

Scheme (1)

Western blot detection can be performed by taking a small amount of tumor tissue from a tumor patient by biopsy or surgery. If the expression of the patient's PDE3A protein is positive (PDE3A+), then Anagrelide can be considered for the treatment, whose effective rate is close to 70%; if the expression of which is PDE3A negative (PDE3A−), the drug is not used, wherein accuracy rate of elimination is 100%.

Scheme (2)

The expression of PDE3A mRNA can be detected by taking a small amount of tumor tissue from a tumor patient by biopsy or surgery, extracting mRNA, and performing RT-PCR reaction. If the expression of PDE3A mRNA is positive (PDE3A+), Anagrelide can be considered for the treatment, wherein the efficiency is close to 70%; if the expression of PDE3A mRNA is negative (PDE3A−), the drug is not used, wherein the accuracy rate of elimination is 100%. For patients with liver cancer, glioma, and cervical cancer, this method can be preferentially considered to be used to detect the PDE3A protein.

Scheme (3)

In the present invention, the expression level of PDE3A in circulating tumor cells in peripheral blood can also be detected.

Detection Kit

The present invention further provides a detection kit for the treatment effect of anagrelide on the tumor, wherein the kit comprises a container a, which contains a PDE3A gene sequence, protein or a specific antibody thereof; and a label or an instruction, The label or instruction indicates that the kit is used for (a) judging the effect of anagrelide on the treatment of a tumor, and/or (b) judging whether a tumor patient is suitable for the treatment with anagrelide. Preferably, containing the anti-PDE3A immunoglobulin or immunoconjugate, or an active fragment thereof.

The Main Advantages of the Present Invention Include:

(a) Providing markers that predict the efficacy of anagrelide on the treatment of tumors, which contributes to the selection of treatment options for patients.

(b) Analyzing the patient's sensitivity to anagrelide in advance to avoid ineffective treatment.

(c) The detection is convenient, fast and economical, which can reduce the cost of patient treatment.

The present invention is further described below with reference to specific embodiments. It should be understood that these examples are only for illustrating the present invention and not intended to limit the scope of the present invention. The experimental methods in the following examples which do not specify the specific conditions are usually carried out according to conventional conditions or according to the conditions recommended by the manufacturer. Unless otherwise indicated, percentages and parts are by weight.

General Material

1. Drugs and Reagents

The drugs and reagents involved in the examples are shown in the following table:

| Name | Company |
|---|---|
| Anagrelide (Cat. No. A637300) | J&K |
| Cilosilostazol (Cat. No. A637300) | SELLECK.CN |
| PDE inhibitors (Cat. No. BML-2842-0500) | Enzo life sciences |
| IBMX (Cat No. I5879) | Sigma-Aldrich Co. |
| Penicilin | Invitrogen(USA) |
| Streptomycin | Invitrogen (USA) |
| DMEM | Invitrogen (USA) |
| RPMI-1640 | Invitrogen (USA) |
| MEM | Invitrogen (USA) |
| Lipofectamine TM2000 | Invitrogen (USA) |
| Fetal Bovine Serum | Gibco |
| Trypsin 1:250 | Genebase, Gene-Tech. |
| | co. Ltd |
| PrimeScript TM RT reagent kit (Code: DRR037S) | Takara |
| SYBR Premix Ex TaqTM(TliRNaseH Plus) kit | Takara |
| (RR420A) | |
| ReverTra Ace reverse transcriptase | TOYOBO |
| TRIzol | Invitrogen (USA) |
| Tris base | Sigma |
| Glycine | Sigma |
| SDS | Sigma |
| BSA | Genebase, Gene-Tech. |
| | co, Ltd |
| MTT | Amresco |
| Laemmli sample buffer | Sigma-Aldrich, Saint |
| | Louis, MO, USA |
| Tween 20 | Sinopharm |
| Protease inhibitors cocktail/PMSF | Sigma |

2. Cell and Cell Culture Medium

The cell and cell culture medium involved in the examples are shown in the following table:

| cell line | source of tissue | culture medium |
|---|---|---|
| A549 | human non-small cell | DMEM + 10% FBS |
| | lung cancer cell | |
| H460 | human non-small cell | PRMI1640 + 10% FBS |
| | lung cancer cell | |
| H522 | human lung cancer cell | PRMI1640 + 10% FBS |
| CASKI | human cervical cancer | PRMI1640 + 10% FBS |
| | epithelial cell | |
| HeLa | human cervical cancer | DMEM + 10% FBS |
| | epithelial cell | |
| MDA-MB-231 | human breast cancer cell | PRMI1640 + 10% FBS |
| MCF-7 | human breast cancer cell | DMEM + 15% FBS |
| MDA-MB-453 | human breast cancer cell | DMEM + 10% FBS |
| U87-MG | human glioma cell | DMEM + 10% FBS |
| H4 | human glioma cell | DMEM + 10% FBS |
| FHCC98 | human hepatoma cell | PRMI1640 + 10% FBS |
| SMMC7721 | human hepatoma cell | PRMI1640 + 10% FBS |
| HepG2 | human hepatoma cell | DMEM + 10% FBS |
| Bel7404 | human hepatoma cell | PRMI1640 + 10% FBS |
| 97H | human hepatoma cell | a-MEM + 10% FBS |
| SKOV3 | human ovarian cancer cell | DMEM + 10% FBS |
| A498 | human kidney cancer cell | PRMI1640 + 10% FBS |
| 786-0 | human kidney cancer cell | PRMI1640 + 10% FBS |
| PC-3 | human prostate cancer cell | PRMI1640 + 10% FBS |
| DU145 | human prostate cancer cell | PRMI1640 + 10% FBS + |
| | | 2 mM Glutamine |
| SW1116 | human colon cancer cell | MEM + 10% FBS |
| WIDR | human colon cancer cell | PRMI1640 + 10% FBS |
| HCT-15 | human colon cancer cell | PRMI1640 + 10% FBS + |
| | | Sodium pyruvate + glucose |
| HCT-116 | human colon cancer cell | PRMI1640 + 10% FBS |
| COLO205 | human colon cancer cell | PRMI1640 + 10% FBS |
| SW480 | human colon cancer cell | PRMI1640 + 10% FBS |
| HT-29 | human colon cancer cell | PRMI1640 + 10% FBS |
| A431NS | human skin cancer cell | DMEM + 10% FBS |

The above cell lines were cultured in a cell incubator containing 5% CO₂ at 37° C., and the cells were subjected to experiments in the logarithmic growth phase.

General Methods

1. Preparation of Tissue Protein Samples

The surgically removed tissue block was quickly placed in pre-cooled 0.95 normal saline, rinsed several times to clean the blood on the surface, the tissue was weighed, cut into several smaller tissue blocks and put into the mechanical tissue homogenizer. According to the the ratio of the net weight of tissue, lysate=1:10, the corresponding volume of lysate was added for homogenization, and the supernatant was collected by centrifugation (If there is a viscous substance, it can be sonicated, or after the nucleic acid was freeze dried and degraded, the lyophilized protein sample can be dissolved into a suitable loading buffer. It is allowed to stand for 3 hours, so that the protein in the sample can be completely dissolved for 5 minutes, and the mixture was centrifuged and collected at 4 degrees.). Laemmli sample buffer (as the protein sample concentration, mixed at the ratio of 1:1 or 1:2). Strongly mixed, the sample was heated in a water bath of 100 degrees for 3-5 minutes, and centrifuged at 10000 g for 10 minutes. The supernatant was removed and transferred to another clean test tube. At this point, the electrophoresis sample was ready (the sample can be immediately used or separately frozen, and the sample stored at −20° C. can be kept stable for several months.)

2. Extraction of Tissue RNA

Tissue (<100 mg) and 1 ml of TRIzol were added into 1.5 ml RNAase-free EP tube, placed on ice for 5 minutes, then 200 μL chloroform was added. The tube was shaked vigorously for 15 s and then put on ice for 3 minutes; the mixture was centrifuged at 12000 g at 4° C. for 15 minutes using low temperature high speed centrifuge, and the upper layer was transferred to a new EP tube; 500 μL of isopropanol was added into the EP tube, and the tube was mixed and placed on ice for 10 minutes; the mixture was centrifuged for at 12000 g for 10 minutes at 4° C. using low-temperature high-speed centrifuge. When white solid was sinked into the bottom of the EP tube, the supernatant was discarded. 1 ml of 75% ethanol (formulated with DEPC water) was added into the EP tube, and the mixture was mixed by shaking; and the mixture was centrifuged at 7500 g in a low-temperature high-speed centrifuge at 4° C. for 5 minutes, then the supernatant was discarded and the ethanol was removed as much as possible; the RNA in the EP tube was placed in the air, and dried to be clear, then was dissolved with 30-50 μL of DEPC water, and stored at −80° C.

3. Determination of Half Poisoning Dose of Drugs to Cells (MTT Method)

The liquid containing drugs was continuously geometrically diluted to 1:10, 1:100, 1:1,000, 1:10,000 with a cell maintenance solution, and a normal cell control group was set up. Different concentrations of each drug were added into a 96-well plate that has been grown into a single layer, respectively, and inoculated per dilution in triplicate, and 100 μl was added per well, and the plate was incubated in a carbon dioxide incubator at 37° C. for 72 h, followed by 5 μg/ml 25 μL of MTT was added.

The culture was continued for 3.5 h, and the supernatant was removed and discarded. 150 μl of the dissolving solution DMSO was added to each well, and the plate was shaked for 5 to 10 min, until the crystals were completely dissolved. Then the OD value at 570 nm was measured by a microplate reader. The cell viability of each drug dilution was calculated: cell viability %=(mean value of experimental well OD measured value/mean value of control well OD measured value)×100%. The drug concentration at 50% cell poisoning (IC50) was calculated using probability unit regression.

4. Western Blot Assay

The separation gel was 8%-10% SDS-polyacrylamide (SDA-PAGE), while the concentrated gel was 5%. The voltage of the concentrated gel was 80V, while the voltage of the separation gel was 120V. The indicator of bromophenol blue was stopped at the bottom; the film was transferred by the semi-dry method, the film transfer voltage was 10-15V, and the film transfer time was adjusted according to the size of the protein molecule; after the film was transferred, the nitrocellulose membrane was pre-dyed with ponceau, and the film was cut. After blocked with TBST containing 5% skim milk for 1 hour on the decolorization shaker, washed with TBST once and the primary antibody was added. The primary antibody was diluted at 1:2000-1:5000 in TBST containing 5% BSA, and the mixture was placed on a decolorizing shaker for 1 hour at room temperature, and then overnight at 4° C. After balancing for 1 hour at room temperature the next day, washed with TBST three times for 10 minutes each time. The secondary antibody was added and the secondary antibody was diluted at 1:3000-1:5000 in TBST containing 5% BSA. The mixture was placed on a decolorization shaker for 1 hour at room temperature, and washed with TBST three times for 10 minutes each time. After exposure to the ECL kit substrate for 2-3 minutes, it was exposed to a chemiluminometer.

5. RT-PCR Reaction

1) Extraction of Total Cellular RNA with Trizol

Single-layer cells and TRIzol 1 ml were placed in a 10 cm diameter small dish; TRIzol was evenly covered in small dish, placed on ice for 5 minutes, then cells were collected and transferred to 1.5 ml RNAase free EP tube; 200 μL chloroform was added into EP tube, shaked vigorously for 15 s and then placed on ice for 3 minutes; the mixture was subjected to 12000 g of low-temperature high-speed centrifuge, and centrifuged at 4° C. for 15 minutes, and the upper layer was transferred to a new EP tube; 500 μL of isopropanol was added into the EP tube, mixed and placed on ice for 10 minutes; the mixture was subjected to 12000 g of low-temperature high-speed centrifuge, and centrifuged at 4° C. for 10 minutes. When white solid was sinked at the bottom of the EP tube, the supernatant was discarded. 1 ml of 75% ethanol (formulated with DEPC water) was added into the EP tube, mixed by shaking; the mixture was subjected to 7500 g of low-temperature high-speed centrifuge, centrifuged at 4° C. for 5 minutes, then the supernatant was discarded, and the ethanol was removed as much as possible; the RNA in the EP tube was placed in the air, and dried to be clear. RNA was dissolved with 30-50 μL of DEPC water, and stored at −80° C.

2) Reverse Transcription Reaction (20 μL Reaction System, Operation on Ice), the Reaction System was Shown as Follows:

| Reagent | Amount of usage | |
|---|---|---|
| RNA | 4-5 | ug |
| DEPC H2O | Add to 10 μL | |
| Primers or OligodT | 1 | μL |
| 70° C. 5 min, 4° C. 5 min | | |
| 5 X Reaction buffer | 4 | μL |
| dNTPs(10 mM) | 2 | μL |
| DEPC H2O | 2 | μL |
| 37° C. 5 min | | |
| ReverTra Ace | 1 | μL |

42° C. 60 min, 70° C. 10 min, 4° C. 5 min

3) Real-Time PCR StepOnePlus™ Real-Time PCR System

The reaction system was shown as follows:

| Reagent | Amount of usage |
|---|---|
| SYBRPremix Ex TaqTM(2×) | 10.0 μL |
| PCR Forward Primer(10 μM) | 0.4 μL |
| PCR Reverse Primer(10 μM) | 0.4 μL |
| ROX Reference Dye(50×) | 0.4 μL |
| DNA template | 2.0 μL |
| dH2O (sterilized distilled water) | 6.8 μL |
| Total | 20.0 μL |

[symbol]

Stage 1: 95° C., 30 s (Replicate:1)

Stage 2: 95° C., 5 s; 60° C., 30 s (Replicate:40)

Stage 3:95° C., 15 s; 60° C., 1 min; 95° C., 15 s (Replicate:1)

Real-time PCR primer sequences are shown as follows:

| Name | Sequence (5′-3′) | |
|---|---|---|
| PDE3A | F: | SEQ ID NO.: 1 |
| | GATGATAAATACGGATGTCTGTC | |
| | R: ACCGCCTGAGGAGCACTAG | SEQ ID NO.: 2 |
| β-Actin | F: TGTCACCAACTGGGACGATA | SEQ ID NO.: 3 |
| | R: GGGGTGTTGAAGGTCTCAAA | SEQ ID NO.: 4 |

According to the analysis software equipped in the StepOnePlus™ Real-Time PCR System (Applied Biosystem) system, the relative expression level of PDE3A was determined according to the 2ΔΔCt method using β-Actin as a reference.

6. RNAi Interference

The usage amount and experimental operation of Lipofectamine™ 2000 refer to the instructions.

The HeLa cells in logarithmic growth phase were inoculated into 96-well plates or RTCA plates at about 6000/well and cultivated overnight until the cells were attached about 70-80%, and the cells were in good condition; a certain amount of serum-free and antibiotic-free medium was added into two EP tubes, respectively, and a suitable amount of siRNA was added into one tube and mixed, while a suitable amount of Lipofectamine™2000 was added into the other tube and mixed at room temperature for 5-10 min. The medium supplemented with Lipofectamine™ reagent was added into the siRNA-containing EP tube and mixed at room temperature for 15 min; during the placement, the cells were washed once with serum-free and non-double antibody medium, and 75 μL of the medium was added, ready for use. After 15 minutes, the mixed medium was added into the cell culture dish, 25 μL/well, and gently shaken and mixed; after cultured at 37° C., 5% CO₂ for 6 hours, the medium was changed to fresh medium containing 10% FBS; after 24-72 h, the expression of RNAi interference protein was detected by western blot.

7. The Monitoring of Cell Growth by Cell Real-Time Monitor (RTCA)

The cells in the logarithmic growth phase were inoculated into a 16 or 96-well plate matched with the cell real-time monitor at 3-7×10⁴/mL per well, and incubated at 37° C. overnight, and then subjected to dosing or transfection. The real-time monitor automatically monitored cell growth for more than 4 days, and the amount of cells was reflected according to the size of the resistance formed by spreading the cells. The larger the Cell Index value was, the faster the cells grew and the more cells were.

8. PI Detection of Apoptotic Cells

In 96-well plates, cells were treated with different compounds for 24-36 hours, and 2 μL of PI dye liquor (25 ug/mL) was added into 96-well plates. Cells were incubated for 20 min, and photographed under a fluorescence microscope for observation of the number of red fluorescent cells.

### Example 1

Detection of PDE3A Protein Expression in Tumor Cell Lines

In order to investigate the expression of PDE3A protein in tumor cells, the expression levels of PDE3A protein were detected by immunoblotting on the cell lines listed in the general materials.

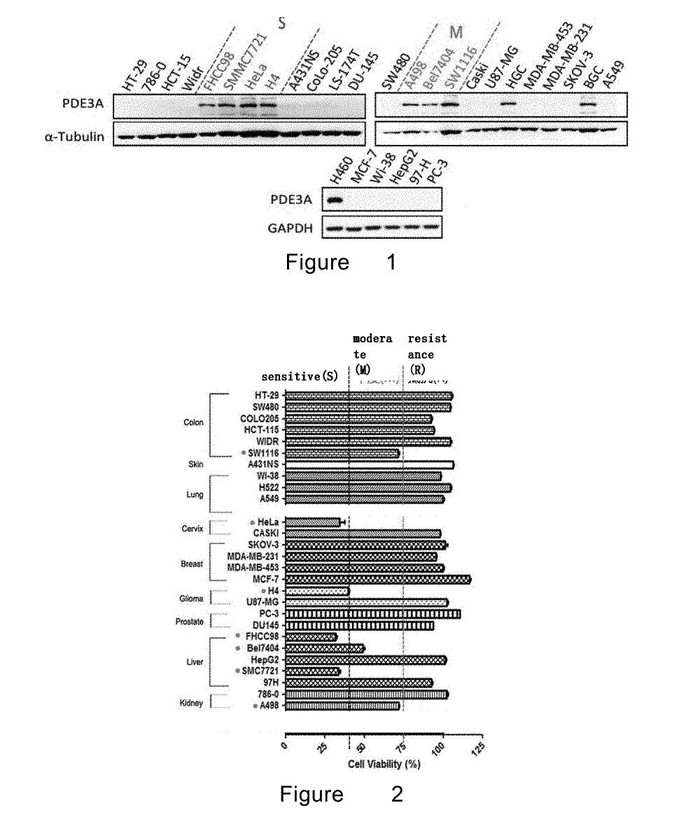

As shown in FIG. 1, the PDE3A protein was positively expressed in SMMC7721, FHCC98, H4, HeLa, Bel7404, A498, SW1116 etc., and negatively expressed in the other 20 cell lines.

Further, PDE3A mRNA levels of some cell lines were detected by RT-PCR; RQ: quantitative results relative to β-actin.

The results were shown in FIG. 3. PDE3A mRNA was expressed in SMMC7721, FHCC98, H4, HeLa, Bel7404, A498 and SW1116. There was almost no PDE3A mRNA expression in H522 and SKOV3, which was consistent with the results of immunoblotting.

### Example 2

Antitumor Activity Assay for Drugs

The cell lines in Example 1 were subjected to an activity assay using Anagrelide at a concentration of 1 μM, and the cell lines were classified into three categories according to cell viability: sensitive (S), moderately sensitive (M), and insensitive (R), and the IC50 value of anagrelide in inhibiting each cell line was further determined.

The results were shown in FIG. 2. There were 7 sensitive cells and 20 insensitive cells in the test cell line (FIG. 2). The IC50 values of anagrelide inhibiting sensitive cell lines were shown in Table 1. Wherein, the cell lines positively expressing PDM3A, such as SMMC7721, FHCC98, H4, and HeLa were anagrelide-sensitive cells, and anagrelide showed an excellent inhibitory effect. The IC50 values were all less than 30 nM. The cell lines positively expressing PDE3A, such as Bel7404, A498 and SW1116 were moderately sensitive cells of anagrelide, and anagrelide had a long-lasting inhibitory effect, and its IC50 value was 0.4 μM-16 μM. Twenty cell lines negatively expressing PDE3A were not sensitive to anagrelide and showed tolerance to anagrelide. Wherein the IC50 values were all >50 μM, i.e., anagrelide could not inhibit their growth.

TABLE 1
IC50 values of 7 sensitive cells

| Cell line | Hela | H4 | FHCC98 | SMMC7721 | Bel7404 | A498 | SW1116 |
|---|---|---|---|---|---|---|---|
| IC50(μM) | 0.022 | 0.023 | 0.008 | 0.015 | 0.428 | 15.900 | 14.510 |

In summary, in PDE3A-positive cells, different degrees of growth inhibition occurred after anagrelide treatment. In addition, none of the PDE3A-negative cells was sensitive to anagrelide.

### Example 3

Effect of PDE3A Protein Knockout on the Growth of Sensitive Cells

Example 1 and Example 2 illustrate the relationship between PDE3A protein and growth inhibition from the perspective of compounds inhibiting protein activity, but the compound may cause a difference in cell selectivity due to its different structure and different mode of action with the target. Therefore, after obtaining PDE3A protein cell expression profile data, the effect of PDE3A protein knockout on cell growth was examined. Two pde3a siRNAs (different efficiencies) were used for the experiment, respectively. 48 hours after siRNA transfection, cells were spreaded on RTCA plates for long-term growth monitoring.

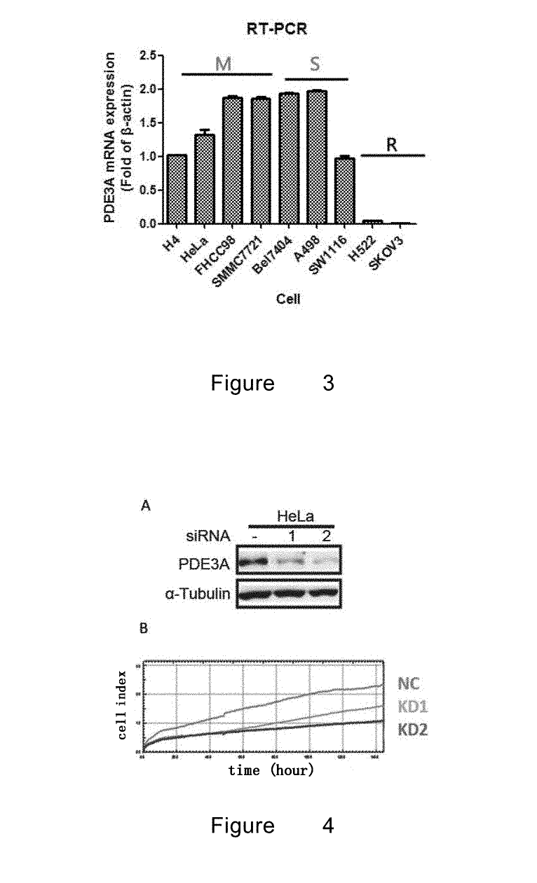

The results were shown in FIG. 4. The expression level of PDE3A protein (FIG. 4A) was positively correlated with the growth rate of cells (FIG. 4B), indicating that PDE3A protein played an important role in the growth of anagrelide-sensitive tumor cells.

### Example 4

The PDE3A Protein was Involved in the Regulation of Apoptosis Induced by Anagrelide

To investigate whether PDE3A protein itself is involved in the regulation of anagrelide-induced apoptosis, firstly siRNA interference technology was used to transfect pde3a siRNA into sensitive cells of HeLa for the knockout, then cells were treated with anagrelide to investigate the relationship between PDE3A protein and cell sensitivity to anagrelide.

Wherein the KD group was HeLa cells transfected with pde3a siRNA, and 1 and 2 represent two siRNAs, respectively.

The NC group was HeLa cells transfected with siRNA without knockout effect, as blank control.

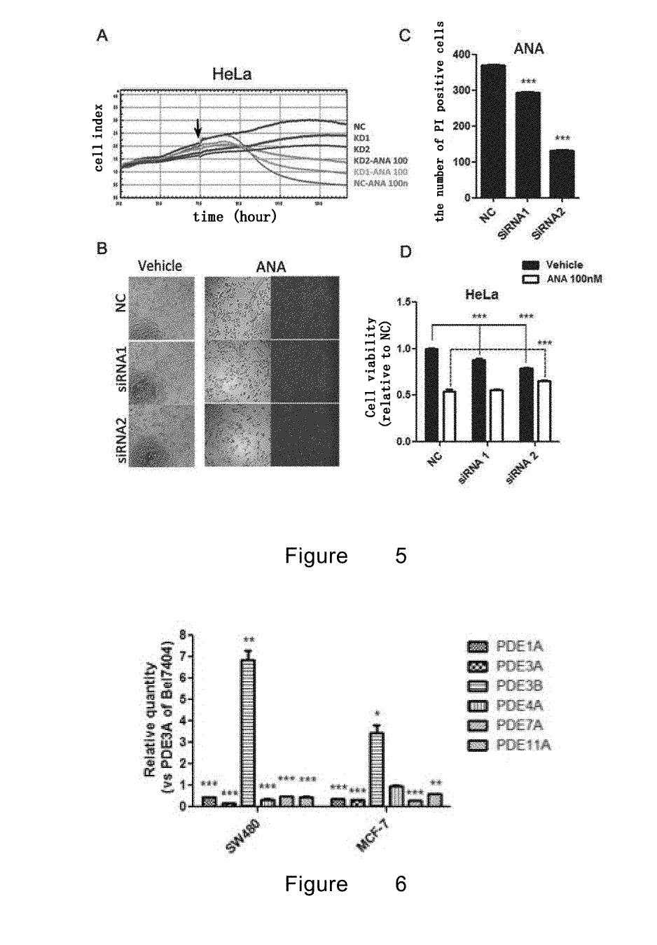

The results showed that the level of PDE3A protein was positively correlated with the growth of HeLa cell without the addition of anagrelide. After anagrelide was added, the survival rate of KD2 with the lower level of PDE3A protein was the highest, while that in the NC group was the lowest (FIG. 5A). 48 hours after PDE3A knockout, HeLa cells were treated with anagrelide for 36 hours, and the cells in the KD group also had fewer PI positive cells than that in the control group (FIG. 5B and FIG. 5C); simultaneous detection by MTT also indicated the lower the PDE3A protein level in HeLa cells, the more favorable it was to resist anagrelide (FIG. 5D). The above results showed that PDE3A protein was indeed involved in the regulation of apoptosis by anagrelide, and the expression level of PDE3A protein can be used as a marker for the therapeutic effect of anagrelide.

### Example 5

Preparation of a Diagnostic Kit for Judging the Treatment Effect of Anagrelide on Tumors

A kit for detecting the effect of anagrelide in treating tumors is prepared, comprising:

(a) a container, and an antibody specific for PDE3A therein: a rabbit anti-PDE3A antibody; and

(b) a label or an instruction indicating that the kit is used to detect the effect of anagrelide in treating a tumor.

### Example 6

Western blotting was used to detect the protein expression levels of other members in the PED family, including PDE1A, PDE3A, PDE3B, PDE4A, PDE7A, and PDE11A, in cell lines of SW480 and MCF-7.

The results were shown in FIG. 6, the expression level of PDE3B in SW480 and MCF-7 was relatively high, while the expression levels of PDE3A and other members of the PED family detected were relatively low.

### DISCUSSION

Anagrelide has been used as a first-line treatment for thrombocytopenia for more than 20 years, while it has always been a blank in the history of solid tumor treatment; previous laboratory studies have shown for the first time that anagrelide can be used clinically as an extremely selective anti-tumor drug. After deepening its mechanism research, it has been realised that PDE3A protein can be used as the first biomarker to assist the diagnosis and treatment of anagrelide in clinical tumor patients. This is the first time that the compound has been researched and has a clear target-assisted individualized tumor treatment. This finding has been confirmed in a series of experiments at the molecular and cellular levels—knockdown of PDE3A protein in sensitive cells using siRNA interference technology, and competitive binding experiments are performed with similar inhibitors, ultimately demonstrating the binding of anagrelide and target protein PDE3A is required for the drug to induce apoptosis in tumor cells.

The present inventors have found that approximately one-third of tumor patients are PDE3A highly-expressed, and PDE3A-positively expressed tumor cells has showed different degrees of sensitivity to anagrelide, whereas PDE3A-negative tumors are not sensitive to anagrelide. Summaries are as follows:

The study has found that anagrelide has showed a strong inhibition on the growth of a variety of tumor cells (liver cancer, melanoma, kidney cancer, colon cancer, glioma, cervical cancer), whereas its mechanism of action has not been revealed. Through the research of the present invention, it has been discovered that the reason why anagrelide has caused cell cycle arrest and apoptosis, and the biomarker of PDE3A protein has been found to guide clinical diagnosis and typing for the individualized targeted therapy for cancer patients.

All publications mentioned herein are incorporated by reference as if each individual document was cited as a reference, as in the present application. It should also be understood that, after reading the above teachings of the present invention, those skilled in the art can make various changes or modifications, equivalents of which falls in the scope of claims as defined in the appended claims.

1. Use of a PDE3A gene sequence, a PDE3A nucleic acid detection reagent, a PDE3A protein, and/or a PDE3A protein detection reagent for preparing a diagnostic reagent or a diagnostic kit, the diagnostic reagent or kit is used for:
(a) judging the effect of anagrelide on the treatment of tumors, and/or
(b) judging whether the tumor patient is suitable for treatment with anagrelide, and/or
(c) judging the sensitivity of tumor cells to anagrelide.

2. The use of claim 1, wherein the judging comprises an auxiliary judgment and/or a prior judgment.

3. The use of claim 1, wherein the sensitivity refers to the sensitivity of the tumor cells in the presence of anagrelide at the following concentrations: 0.001 to 0.25 μM, preferably 0.01 to 0.1 μM, more preferably 0.02 to 0.08 μM.

4. The use of claim 1, wherein the PDE3A nucleic acid detection reagent or PDE3A protein detection reagent is coupled or carried with a detectable label.

5. The use of claim 1, wherein the detectable label is selected from the group consisting of: a chromophore, a chemiluminescent group, a fluorophore, an isotope or an enzyme.

6. The use of claim 1, wherein the diagnostic reagent or diagnostic kit is used to detect a sample selected from the group consisting of: a surgically removed tissue sample, a biopsy puncture tissue sample, a tumor tissue lysate, a blood sample, a cell sample, a body fluid sample, a urine sample, and a combination thereof.

7. A diagnostic kit for detecting the effect of anagrelide on the treatment of tumor, comprising:
(a) a first container containing a detection reagent for detecting PDE3A expression and/or PDE3A protein;
(b) a label or an instruction; and
(c) optionally a standard or reference substance,
wherein the label or the instruction indicates that the kit is used for (a) judging the effect of anagrelide on the treatment of tumor, and/or (b) judging whether a tumor patient is suitable for the treatment with anagrelide.

8. The kit of claim 7, wherein the label or the instruction indicates the following contents:
(i) PDE3A expression is positive in tumor tissues, and the predictions suggest that anagrelide is effective in treating the tumor, and/or the tumor patients are suitable for the treatment with anagrelide; and
(ii) PDE3A expression is negative in tumor tissues, and the predictions suggest that anagrelide is poorly effective in treating the tumor, and/or that tumor patients are not suitable for the treatment with anagrelide.

9. An in vitro method for judging the sensitivity of a tumor cell to anagrelide, comprising the steps of:
(i) providing a tumor cell to be tested;
(ii) detecting the expression and/or activity of PDE3A in the tumor cell to be tested in vitro, wherein if the tumor cell to be tested expresses PDE3A and/or has PDE3A protein activity, indicating that the tumor cell to be tested is sensitive to anagrelide; if the tumor cell to be tested has low or no expression of PDE3A and/or does not have PDE3A protein activity, indicating that the tumor cell to be tested is not sensitive to anagrelide.

10. The method of claim 9, which further comprises:
(iii) in the presence of anagrelide, in vitro culturing the tumor cell to be tested which is determined to be sensitive to anagrelide in the previous step, and observing the growth condition of the tumor cell, thereby verifying the sensitivity of the tumor cell to anagrelide.


SEQUENCE LISTING (4 sequences)

SEQ ID NO: 1 - DNA, 23 bp, Artificial Sequence
  Synthetic polynucleotide
gatgataaatacggatgtctgtc

SEQ ID NO: 2 - DNA, 19 bp, Artificial Sequence
  Synthetic polynucleotide
accgcctgaggagcactag

SEQ ID NO: 3 - DNA, 20 bp, Artificial Sequence
  Synthetic polynucleotide
tgtcaccaactgggacgata

SEQ ID NO: 4 - DNA, 20 bp, Artificial Sequence
  Synthetic polynucleotide
ggggtgttgaaggtctcaaa